(19) United States
(12) Patent Application Publication
(10) Pub. No.: US 2025/0352447 A1
(43) Pub. Date: Nov. 20, 2025

(54) COMPOSITION FOR CARING FOR THE SKIN

(71) Applicant: L'OREAL, Paris (FR)

(72) Inventors: Yuanji GUO, Shanghai (CN); Xiuxia WANG, Shanghai (CN); Ning CHANG, Shanghai (CN)

(73) Assignee: L'OREAL, Paris (FR)

(21) Appl. No.: 18/870,590

(22) PCT Filed: Jul. 1, 2022
(86) PCT No.: PCT/CN2022/103230

§ 371 Date: Nov. 29, 2024

(51) Int. Cl. A61K 8/37; A61K 8/92; A61Q 19/00

(52) U.S. Cl. CPC A61K 8/375; A61K 8/922; A61Q 19/00; A61K 2800/31

(57) ABSTRACT

It relates to an anhydrous composition for caring for the skin, in particular the scalp, comprising: (i) at least one ester oil of fatty acid containing 14-20 carbon atoms and glycerol; (ii) at least one ester oil of fatty acid containing 6-10 carbon atoms and glycerol; and (iii) a combination of hydrogenated rapeseed oil and hydrogenated palm oil, wherein the weight ratio of hydrogenated rapeseed oil to hydrogenated palm oil is from 45:55 to 90:10. It also relates to a non-therapeutic method for caring for the skin, in particular the scalp, comprising applying the composition to the skin, in particular the scalp.

### TECHNICAL FIELD

The present invention relates to a composition. In particular, the present invention relates to a composition for caring for the skin, in particular the scalp. The present invention also relates to a non-therapeutic process for caring for the skin, in particular the scalp.

### BACKGROUND ART

The skin constitutes a physical barrier between the body and its surroundings. It is constituted of two tissues: the epidermis and the dermis.

The dermis provides the epidermis with a solid support. It is also its nourishing element. It is mainly constituted of fibroblasts and an extracellular matrix, which is composed mainly of collagen, elastin and a substance known as ground substance, these components being synthesized by the fibroblast. Leukocytes, mast cells or else tissue macrophages are also found therein. It also contains blood vessels and nerve fibers.

The epidermis is a desquamating pluristratified epithelium of 100 μm thick on average and is conventionally divided into a basal layer of keratinocytes constituting the germinal layer of the epidermis, a spinous layer constituted of several layers of polyhedral cells positioned on the germinal cells, a granular layer constituted of flattened cells containing distinct cytoplasmic inclusions, keratohyalin granules, and finally an upper layer known as the cornified layer (or stratum corneum), constituted of keratinocytes at the terminal stage of their differentiation, known as corneocytes. These are mummified anucleate cells which derive from keratinocytes. The stack of these corneocytes constitutes the cornified layer that is responsible, inter alia, for the barrier function of the epidermis, i.e. it constitutes a barrier against external attacks, especially chemical, mechanical or infectious attacks and it also makes it possible to protect the body from water loss.

In order to change the appearance of the hair, there are many products developed to treat the hair during dyeing, bleaching, waving and/or straightening.

During the application of products for dyeing the hair, the scalp might be stained by the products applied.

Thus, there is a need to formulate a cosmetic composition for caring for the skin, in particular the scalp, which can prevent the scalp from being stained.

In addition, it is also desired that the composition is stable and has a suitable viscosity.

### SUMMARY OF THE INVENTION

An object of the present invention is to provide a composition for caring for the skin, in particular the scalp, which can prevent the skin, in particular the scalp from being stained, and is stable and has a suitable viscosity.

Accordingly, in a first aspect, the present invention provides an anhydrous composition for caring for the skin, in particular the scalp, comprising:
(i) at least one ester oil of fatty acid containing 14-20 carbon atoms and glycerol;
(ii) at least one ester oil of fatty acid containing 6-10 carbon atoms and glycerol; and
(iii) a combination of hydrogenated rapeseed oil and hydrogenated palm oil,
wherein the weight ratio of hydrogenated rapeseed oil to hydrogenated palm oil is from 45:55 to 90:10.

The composition of the present invention can be in the form of oil gel or oil balm.

In a second aspect, the present invention provides a non-therapeutic method for caring for the skin, in particular the scalp, comprising applying the composition according to the first aspect of the present invention to the skin, in particular the scalp.

It was found that the composition according to the present invention can avoid the scalp being stained by treatment on the hair.

It was also found that the composition according to the present invention is stable and has a suitable viscosity.

Other subjects and characteristics, aspects and advantages of the present invention will emerge even more clearly on reading the detailed description and the examples that follow.

### DETAILED DESCRIPTION OF THE INVENTION

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well as optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

All percentages in the present invention refer to weight percentage, unless otherwise specified.

For the purposes of the present invention, the term “anhydrous” means that the composition according to the present invention contains less than 2 wt. % and preferably less than 0.5 wt. % of water relative to the total weight of the composition. Where appropriate, such small amounts of water may be provided by ingredients of the composition that contain it in residual amount but are not deliberately provided. In particular, there is no water in the composition.

According to the first aspect, the present invention provides an anhydrous composition for caring for the skin, in particular the scalp, comprising:
(i) at least one ester oil of fatty acid containing 14-20 carbon atoms and glycerol;
(ii) at least one ester oil of fatty acid containing 6-10 carbon atoms and glycerol; and
(iii) a combination of hydrogenated rapeseed oil and hydrogenated palm oil;
wherein the weight ratio of hydrogenated rapeseed oil to hydrogenated palm oil is from 45:55 to 90:10.

### Ester Oil of Fatty Acid Containing 14-20 Carbon Atoms and Glycerol

According to the first aspect, the composition according to the present invention comprises at least one ester oil of fatty acid containing 14-20 carbon atoms and glycerol.

The fatty acid containing 14-20 carbon can be saturated or unsaturated. As examples of fatty acid containing 14-20 carbon, mention can be made of palmitic acid, stearic acid, and arachidic acid.

Advantageously, the fatty acid contains 14-18 carbon atoms, preferably 16-18 carbon atoms.

Preferably, the ester oil of fatty acid containing 14-20 carbon atoms and glycerol is selected from a vegetable oil.

More preferably, the ester oil of fatty acid containing 14-20 carbon atoms and glycerol is selected from vegetable oils comprising at least one ester oil of fatty acid containing 14-20 carbon atoms, preferably, 16-18 carbon atoms, and glycerol, such as sunflower seed oil, corn oil, soybean oil, marrow oil, grapeseed oil, sesame seed oil, hazelnut oil, apricot oil, macadamia oil, castor oil, olive oil; avocado oil; argan oil; camellia oil, palm oil, maize germ oil, cottonseed oil, peanut oil, pumpkin seed oil, wheatgerm oil, babassu oil, coconut oil, rapeseed oil, almond oil, linseed oil, safflower oil, and mixtures thereof.

Most preferably, the ester oil of fatty acid containing 14-20 carbon atoms and glycerol is selected sunflower seed oil, jojoba seed oil, argania spinosa kernel oil, coconut oil, corn oil, soybean oil, marrow oil, grapeseed oil, sesame seed oil, hazelnut oil, apricot oil, macadamia oil, castor oil, and avocado oil.

As commercial products of vegetable oil suitable for the composition according to the present invention, mention can be made of Helianthus annuus (sunflower) seed oil sold under the name REFINED SUNFLOWER OIL by the company AAK KAMANI PRIVATE, SIMMONDSIA CHINENSIS (JOJOBA) SEED OIL sold under the name JD FFL JOJOBA GOLDEN OIL by the company JOJOBA DESERT, Cocos nucifera (coconut) oil sold under the name ACTIVATED VIRGIN COCONUT OIL by the company BIOTROPICS, ARGANIA SPINOSA KERNEL OIL sold under the name LIPOFRUCTYL ARGAN BE LS 9779 by the company BASF BEAUTY CARE SOLUTION, and prunus armeniaca (apricot) kernel oil sold under the name APRICOT KERNEL OIL REFINED DAC by the company GUSTAV HEESS.

Advantageously, the ester oil of fatty acid containing 14-20 carbon atoms and glycerol is present in an amount ranging from 10 wt. % to 90 wt. %, preferably from 30 wt. % to 80 wt. %, more preferably from 60 wt. % to 80 wt. %, relative to the total weight of the composition.

### Esters Oil of Fatty Acid Containing 6-10 Carbon Atoms and Glycerol

According to the first aspect, the composition according to the present invention comprises at least one ester oil of fatty acid containing 6-10 carbon atoms and glycerol.

The fatty acid containing 6-10 carbon atoms for the ester oil can be saturated or unsaturated, linear or branched.

For example, the fatty acid can be hexanoic acid, heptanoic acid, caprylic acid, nonanoic acid, capric acid and a combination thereof.

In some embodiments, the at least one ester oil of fatty acid containing 6-10 carbon atoms and glycerol is selected from triglycerides of hexanoic acid, heptanoic acid, caprylic acid, nonanoic acid, capric acid, and a combination thereof.

Preferably, the ester oil of fatty acid containing 6-10 carbon atoms and glycerol is caprylic/capric triglyceride.

As commercial product of caprylic/capric triglyceride, mention can be made of that sold under the name MASESTER E7000 LR01-1/MB by the company PT MUSIM MAS.

Advantageously, the ester oil of fatty acid containing 6-10 carbon atoms and glycerol is present in an amount ranging from 5 wt. % to 35 wt. %, preferably from 10 wt. % to 30 wt. %, more preferably from 15 wt. % to 20 wt. %, relative to the total weight of the composition.

### Hydrogenated Oils

According to the first aspect, the composition according to the present invention comprises a combination of hydrogenated rapeseed oil and hydrogenated palm oil.

For the purpose of the present invention, the weight ratio of hydrogenated rapeseed oil to hydrogenated palm oil is from 45:55 to 90:10.

Preferably, the weight ratio of hydrogenated rapeseed oil to hydrogenated palm oil is from 50:50 to 80:20.

More preferably, the weight ratio of hydrogenated rapeseed oil to hydrogenated palm oil is from 50:50 to 70:30.

In some embodiments, the composition according to the present invention comprises a combination of hydrogenated rapeseed oil and hydrogenated palm oil, wherein the weight ratio of hydrogenated rapeseed oil to hydrogenated palm oil is 50:50.

As commercial product of a combination of hydrogenated rapeseed oil and hydrogenated palm oil, wherein the weight ratio of hydrogenated rapeseed oil to hydrogenated palm oil is 50:50, mention can be made of that sold under the name DERMOFEEL VISCOLID MB by the company EVONIK.

Advantageously, the combination of hydrogenated rapeseed oil and hydrogenated palm oil is present in an amount ranging from 1 wt. % to 20 wt. %, preferably from 3 wt. % to 17 wt. %, more preferably from 5 wt. % to 10 wt. %, relative to the total weight of the composition.

### Additional Adjuvants or Additives

The composition of the present invention may comprise conventional cosmetic adjuvants or additives, for instance fragrances, chelating agents (for example, disodium EDTA), preserving agents (for example, chlorphenesin and phenoxyethanol) and bactericides, fillers, pH regulators (for example citric acid, sodium hydroxide, potassium hydroxide), and mixtures thereof.

The skilled in the art can select the amount of the additional adjuvants or additive so as not to adversely affect the final use of the composition according to the present invention.

According to a particularly preferred embodiment, the present invention provides an anhydrous composition for caring for the skin, in particular the scalp, comprising, relative to the total weight of the composition:
(i) from 60 wt. % to 80 wt. % of at least one vegetable oil comprising at least one ester oil of fatty acid containing 14-20 carbon atoms and glycerol;
(ii) from 15 wt. % to 20 wt. % of caprylic/capric triglyceride; and
(iii) from 5 wt. % to 10 wt. % of a combination of hydrogenated rapeseed oil and hydrogenated palm oil,
wherein the weight ratio of hydrogenated rapeseed oil to hydrogenated palm oil is from 50:50 to 70:30.

### Galenic Form and Use

The composition of the present invention can be in the form of oil gel or oil balm.

The viscosity of the composition according to the invention can be measured at 25° C., using a ProRheo R180 viscometer equipped with a spindle M4 rotating at 200 rpm.

According to a preferred embodiment, the composition of the present invention has a viscosity of from 2.4*10³ mpa·s to 19.2*10³ mpa·s preferably from 2.4*10³ mpa·s to 12*10³ mpa·s as measured at 25° C. using a Rheomat R180 viscometer equipped with a spindle M4 rotating at 200 rpm.

The composition according to the present invention is stable, i.e., the composition can keep unchanged for at least 2 months at 4° C. or 45° C., without oil leakage.

According to the second aspect, the present invention provides a non-therapeutic method for caring for the skin, in particular the scalp, comprising applying the composition according to the first aspect of the present invention to the skin, in particular the scalp.

The composition according to the present invention can prevent the skin, in particular the scalp from being stained.

The following examples serve to illustrate the present invention without, however, being limiting in nature.

### Examples

Main raw materials used, trade names and supplier thereof are listed in Table 1.

TABLE 1

| INCI Name | Trade Name | Supplier |
|---|---|---|
| HELIANTHUS ANNUUS | REFINED | AAK KAMANI |
| (SUNFLOWER) SEED OIL | SUNFLOWER OIL | PRIVATE |
| CAPRYLIC/CAPRIC | MASESTER E7000 | PT MUSIM |
| TRIGLYCERIDE | LR01-1/MB | MAS |
| HYDROGENATED | CEGESOFT HF 52 | BASF |
| VEGTABLE OIL | | |
| SYNTHETIC WAX | PERFORMALENE | NUCERA |
| | 500-L | SOLUTIONS |
| | POLYETHYLENE | |
| HYDROGENATED | DERMOFEEL | EVONIK |
| RAPESEED OIL (and) | VISCOLID MB | |
| HYDROGENATED PALM OIL | | |

Invention Example (IE.1) 1 and Comparative Examples 1-3 Compositions according to invention example 1 (IE) 1 and comparative examples (CE) 1-3 were prepared according to the contents given in Table 2 (the contents are expressed as weight percentages of active material relative to the total weight of each composition, unless otherwise indicated).

TABLE 2

| | IE. 1 | CE. 1 | CE. 2 | CE.3 |
|---|---|---|---|---|
| Components | wt. % | wt. % | wt. % | wt. % |
| HELIANTHUS ANNUUS | QS100 | QS100 | QS100 | QS100 |
| (SUNFLOWER) SEED OIL | | | | |
| CAPRYLIC/CAPRIC | 15 | 15 | 15 | 15 |
| TRIGLYCERIDE | | | | |
| HYDROGENATED | 5 | — | — | — |
| RAPESEED OIL (and) | | | | |
| HYDROGENATED PALM OIL* | | | | |
| HYDROGENATED | — | 5 | — | — |
| VEGTABLE OIL** | | | | |
| SYNTHETIC WAX | — | — | 5 | — |

*the weight ratio of hydrogenated rapeseed oil to hydrogenated palm oil is 50:50.
**a combination of hydrogenated rapeseed oil and hydrogenated palm oil wherein the weight ratio of hydrogenated rapeseed oil to hydrogenated palm oil is 40:60.

### Preparation Process:

The compositions listed above were prepared as follows, taking the composition of invention example 1 as an example:
1). Mixing HELIANTHUS ANNUUS (SUNFLOWER) SEED OIL and CAPRYLIC/CAPRIC TRIGLYCERIDE to obtain an oily phase and heating the mixture to 80° C. and homogenising the oily phase by stirring;
2). Adding HYDROGENATED RAPESEED OIL (and) HYDROGENATED PALM OIL into the oily phase and homogenising by stirring; and
3). Discharging at 35° C.

The composition of invention example 1 is an oil gel.

### Evaluation

Viscosity, stability, and protection ability of each composition prepared above were characterized.

### Viscosity

The viscosity was measured at 25° C., using a Rheomat R180 viscometer equipped with a M4 spindle, the measurement being performed after 30 s of rotation of the spindle in the composition (after which time stabilization of the viscosity and of the spin speed of the spindle are observed), at a shear rate of 200 rpm.

The results on viscosity were summarized in Table 3.

TABLE 3

| Properties | IE. 1 | CE. 1 | CE. 2 |
|---|---|---|---|
| Viscosity | 15 UD | 10 UD | 25 UD |
| | 3.6*10³ mpa · s | 2.4*10³ mpa · s | 6*10³ mpa · s |

### Stability

The appearance of each composition prepared above upon storage at 4° C. or 45° C. for 2 months was observed to evaluate the stability of the composition.

The results on stability were summarized in Table 4.

TABLE 4

| Properties | | IE. 1 | CE. 1 | CE. 2 |
|---|---|---|---|---|
| Stability | 4° C. | No oil leakage | No oil leakage | Oil leakage |
| | 45° C. | | Oil leakage | No oil leakage |

### Protection Ability

The in-vitro protection function of the compositions of invention example 1 and comparative examples 1-3 were evaluated by as follows.
1. A pig skin of 4 cm² was washed with a normal shampoo without conditioning, and dried.
2. The composition to be tested was applied at 30μ/cm² on the pig skin,
3. Then an oxidative hair coloration product (a mixture of a black coloration cream and a developer comprising 30 vol H₂O₂ mixed at a volume ratio of 1:1.5) was applied at
75μ/cm² on the pig skin for 30 minutes,
4. The skin was rinsed off with the normal shampoo without conditioning,
5. Images were collected and the scalp stain was evaluated.

TABLE 5

| | Ngc* | IE. 1 | CE. 1 | CE. 2 | CE. 3 |
|---|---|---|---|---|---|
| scalp stain (%) | 90 | 10 | 30 | 50 | 70 |

Ngc* means there is no protection composition applied.
80%~100% scalp stain indicates fully black stain on the pig skin.
60%~80% scalp stain indicates majority black stain on the pig skin.
40~60% scalp stain indicates about half of black stain on the pig skin.
10~30% scalp stain indicates less black stain on the pig skin.

It can be seen from Table 5 that the composition of invention example 1 can provide a better protection as compared with compositions of comparative examples 1-3.

1: An anhydrous composition for caring for the skin, comprising:
(i) at least one ester oil of a fatty acid containing 14-20 carbon atoms and glycerol;
(ii) at least one ester oil of a fatty acid containing 6-10 carbon atoms and glycerol; and
(iii) a combination of hydrogenated rapeseed oil and hydrogenated palm oil,
wherein a weight ratio of hydrogenated rapeseed oil to hydrogenated palm oil is from 45:55 to 90:10.

2: The composition according to claim 1, wherein the ester oil of a fatty acid containing 14-20 carbon atoms and glycerol is selected from the group of vegetable oils consisting of sunflower seed oil, jojoba seed oil, argania spinosa kernel oil, coconut oil, corn oil, soybean oil, marrow oil, grapeseed oil, sesame seed oil, hazelnut oil, apricot oil, macadamia oil, castor oil, and avocado oil.

3: The composition according to claim 1, wherein the ester oil of a fatty acid containing 14-20 carbon atoms and glycerol is present in an amount of from 10 wt. % to 90 wt. relative to the total weight of the composition.

4: The composition according to claim 1, wherein the fatty acid containing 6-10 carbon atoms for the ester oil can be saturated or unsaturated, linear or branched.

5: The composition according to claim 1, wherein the at least one ester oil of a fatty acid containing 6-10 carbon atoms and glycerol is selected from the group consisting of triglycerides of hexanoic acid, triglycerides of heptanoic acid, triglycerides of caprylic acid, triglycerides of nonanoic acid, triglycerides of capric acid, and a combination thereof.

6: The composition according to claim 1, wherein the ester oil of a fatty acid containing 6-10 carbon atoms and glycerol is present in an amount of from 5 wt. % to 35 wt. %, relative to the total weight of the composition.

7: The composition according to claim 1, wherein the weight ratio of hydrogenated rapeseed oil to hydrogenated palm oil is from 50:50 to 80:20.

8: The composition according to claim 1, wherein the combination of hydrogenated rapeseed oil and hydrogenated palm oil is present in an amount of from 1 wt. % to 20 wt. %, relative to the total weight of the composition.

9: The cosmetic composition according to claim 1 comprising, relative to the total weight of the composition:
(i) from 60 wt. % to 80 wt. % of at least one vegetable oil comprising at least one ester oil of a fatty acid containing 14-20 carbon atoms and glycerol;
(ii) from 15 wt. % to 20 wt. % of caprylic/capric triglyceride; and
(iii) from 5 wt. % to 10 wt. % of a combination of hydrogenated rapeseed oil and hydrogenated palm oil,
wherein the weight ratio of hydrogenated rapeseed oil to hydrogenated palm oil is from 50:50 to 70:30.

10: The composition according to claim 1, having a form of an oil gel or an oil balm.

11: A non-therapeutic method for caring for the skin, comprising applying the composition according to claim 1, to the skin.

12: The non-therapeutic method for caring for the skin according to claim 11, wherein the composition is applied to the scalp.
